(19) United States
(12) Patent Application Publication
(10) Pub. No.: US 2016/0202186 A1
(43) Pub. Date: Jul. 14, 2016

(54) Gas Sensor to Enhance Implementation of a Process-Based Leakage Monitoring Method

(71) Applicants: BOARD OF REGENTS, THE UNIVERSITY OF TEXAS SYSTEM, Austin, TX (US); MESA PHOTONICS, LLC, Santa Fe, NM (US)

(72) Inventors: Katherine Romanak, Austin, TX (US); David Bomse, Santa Fe, NM (US); Marwood N. Ediger, Albuquerque, NM (US)

(21) Appl. No.: 14/912,131

(22) PCT Filed: Aug. 15, 2014
(86) PCT No.: PCT/US2014/051179

§ 371 Date: Feb. 15, 2016

Related U.S. Application Data
(60) Provisional application No. 61/866,578, filed on Aug. 16, 2013.

(51) Int. Cl. G01N 21/65; G01N 30/16; G01N 33/00

(52) U.S. Cl. CPC G01N 21/65; G01N 33/0036; G01N 30/16; G01N 2201/06113; G01N 2201/08

(57) ABSTRACT

The present invention includes an apparatus (10) and method for detecting nitrogen gas comprising: a conduit (12) for a gas sample (16); one or more hollow core photonic crystal fibers HC-PCF (20) having a proximal portion and a distal portion, wherein the proximal portion is in communication with the gas sample (16) in the conduit (12); a laser (14) positioned to strike the gas sample (16) in the conduit (12) and opposite the one or more hollow core photonic crystal fibers (20); and a Raman spectra detector (30) connected to the distal portion of the hollow core photonic crystal fibers (20), wherein a Raman spectra is generated when the laser (14) strikes the gas sample (16) that is detected by the Raman spectra detector (30).

### TECHNICAL FIELD OF THE INVENTION

The present invention relates in general to the field of gas detection, and more particularly, to a novel Nitrogen sensor to enhance implementation of a process-based leakage monitoring method.

### BACKGROUND OF THE INVENTION

Without limiting the scope of the invention, its background is described in connection with detection of gases.

Carbon capture and storage (CCS) relies to a large extent on existing infrastructure and established oil-field technology. CCS can also make it practical for the U.S. to continue using coal for electricity generation. Reliable implementation of CCS requires trustworthy measurement, monitoring and verification (MMV) technologies. For example, standards set the by U.S. Environmental Protection Agency (EPA) require that 99% of stored carbon dioxide remain underground. MMV can utilize many techniques deployed at a range of depths from the storage reservoir to the atmosphere; however, techniques that monitor leakage through the near surface vadose zone are valuable because: (1) the vadose zone is the interface between subsurface storage and release to atmosphere, (2) gases moving through the shallow subsurface can be monitored easily and cheaply, and (3) vadose zone monitoring can directly address concerns of landowners living above CCS sites. Measurements of carbon dioxide concentrations alone, however, are not effective for CCS leak detection. High variability of CO₂ generated in situ can mask a moderate leak. Background measurements require a long lead time (a year or more) potentially hindering a project's progress, and background CO₂ in soil gas cannot be measured practically over a sufficiently fine grid to be useful for identifying small leaks. We follow a process-based leak detection approach that characterizes gas concentrations in monitoring wells by considering atmospheric, geochemical, and biological processes that occur in soil. Additional CO₂, not accounted for by these processes, is identified as having an exogenous source.

Measurements of the amounts of four gases within the soil are used to determine the active process or processes within the soil and to identify the amount of exogenous CO₂ that may be present. The four gases are carbon dioxide (CO₂), oxygen (O₂), nitrogen (N₂), and methane (CH₄). Water vapor concentrations may also be measured or can be estimated based on inferences about the soil gas humidity. Gas chromatography (GC) has been used in research studies of soil gases because it is sensitive to all four gases over the ranges of concentrations needed for MMV. Adapting GC for continuous measurements of soil gas is made difficult by the need for a reliable supply of a carrier gas such as helium. A variety of easily-implemented and commercially-available methods exist for measuring the amounts of CO₂, O₂, and CH₄ within gas mixtures. In contrast, few methods in addition to GC exist for N₂ concentration measurements.

Raman spectroscopy is an optical technique that can be used to measure N₂ concentrations and N₂ number densities in gas mixtures. The Raman effect is weak, however. For example, when a 1 W, 488 nm wavelength light beam in incident on air at 1 atm and 0° C., only 4.4 billionths of a Watt of Raman light is emitted per cm of optical path. The Raman light is emitted isotropically; only a small fraction of the emitted light can be collected and analyzed. Attempted improvements include increasing the incident light power by placing the gas sample inside the cavity of a laser, placing the sample within an optical cavity external to the laser, and using a multiple pass optical cell. None are suitable for routine MVA measurements.

The amount of collected Raman light can be increased by placing the gas cell within an optical waveguide such as a hollow-core photonic crystal fiber. Confining much of the laser power within the fiber core increases the amount of incident light in the gas. The waveguiding properties of the fiber also transport a significant portion of the emitted Raman light to the ends of the fiber from which the light is emitted into a cone defined by the numerical aperture of the fiber. Prior art demonstrates the waveguide approach for gases including N₂, O₂, and H₂.

However, the current patents and references teach against the use of Raman spectroscopy as it relates to CO₂ and CH₄. The Raman cross sections for CO₂ and CH₄ are over ten orders of magnitude smaller (i.e., weaker) than the infrared absorption cross sections for the gases. Hence, the preference for absorption-based sensing for CO₂ and CH₄ and the fact that useful Raman detection of the two gases is novel.

The prior art includes a patent that was issued to Chen, et al, entitled “Hollow-core waveguide-based Raman systems and methods,” U.S. Pat. No. 7,595,882, which teaches a system for sensing homonuclear diatomic molecules, such as, for example, nitrogen. Some embodiments are said to include a method for sensing homonuclear diatomic molecules using a system that includes a light source, a hollow-core wave-guiding device that exhibits a low attenuation at predetermined operating optical frequencies and is in optical communication with the light source, a gas introduction system for introducing a gaseous medium between the light source and the hollow-core wave-guiding device, and a detector in optical communication with the hollow-core wave-guiding device.

Another such patent issued to Dong and Peng, entitled “Photonic Bandgap Fibers,” U.S. Pat. No. 7,209,619, which teaches photonic bandgap fibers designed to provide a desired dispersion spectrum. The patent is said to teach designs for achieving wide transmission bands and lower transmission loss. One such example, in some fiber designs, uses smaller dimensions of high index material in the cladding and large core size provide small flat dispersion over a wide spectral range. Another example uses the thickness of the high index ring-shaped region closest to the core has sufficiently large dimensions to provide negative dispersion or zero dispersion at a desired wavelength. Additionally, low index cladding features are said to be distributed along concentric rings or circles may be used for achieving wide bandgaps.

Other background references include, e.g., Buric et al, “Enhanced spontaneous Raman Scattering and gas composition analysis using a photonic crystal fiber,” Appl Opt 47, 4255-4261 (2008); and Chen et al, “Photonic bandgap fiber-enabled Raman detection of nitrogen gas,” SPIE vol. 7322 (2009).

### SUMMARY OF THE INVENTION

The present invention includes a gas analyzer that may be used as part of a soil gas analysis method that can identify carbon dioxide leaking from underground carbon capture and storage (CCS) sites and potentially at shale gas sites. The gas analysis method at CCS sites requires determining the concentrations in the soil gas of carbon dioxide (CO₂), oxygen (O₂), methane (CH₄), water vapor (H₂O), and nitrogen (N₂). As practiced, CO₂, O₂, CH₄, and N₂ concentrations are measured using Raman scattering from the soil gas when soil gas samples are contained within the core of a hollow core photonic crystal fiber (HC-PCF). Water vapor is measured using a commercial humidity sensor. Raman scattering is a weak optical process and, using typical optical configurations, Raman signals from gases are difficult to detect. The spatial and optical properties of hollow core photonic crystal fibers combine to increase Raman intensity by up to a factor of 1,000 over conventional Raman implementations. This signal increase is necessary to achieve useful accuracy and precision of gas concentrations when used as part of soil gas analysis.

The present invention provides a gas analyzer having a conduit for a gas sample; one or more hollow core photonic crystal fibers (HC-PCF) having a proximal portion and a distal portion, wherein the proximal portion is in communication with the gas sample in the conduit; a laser positioned to strike the gas sample in the conduit and opposite the one or more hollow core photonic crystal fibers; and a Raman spectra detector connected to the proximal portion of the hollow core photonic crystal fibers, wherein a Raman spectrum is generated when the laser strikes the gas sample that is detected by the Raman spectra detector without regard to the level of attenuation. The detector may be a spectrometer and may be adapted for linear Raman spectroscopy. The detector may include one or more avalanche photodiodes. The gas may be at a pressure at or about atmospheric pressure, above atmospheric pressure, or below atmospheric pressure. The analyzer may not require any consumable supplies. The apparatus may further include a microscope objective between the laser and the Raman detector and/or at least one of a power source, a small solar panel, a storage battery, data storage, instrument diagnostics, and wireless capabilities to transmit data via a wireless network.

The present invention provides a method of for detecting N₂ including obtaining a sample suspected of comprising N₂; and injecting the sample into an N₂ analyzed comprising: a conduit for a gas sample; one or more hollow core photonic crystal fibers (HC-PCF) having a proximal portion and a distal portion, wherein the proximal portion is in communication with the gas sample in the conduit; a laser positioned to strike the gas sample in the conduit and opposite the one or more hollow core photonic crystal fibers; and a Raman spectra detector connected to the proximal portion of the hollow core photonic crystal fibers, wherein a Raman spectra is generated when the laser strikes the gas sample that is detected by the Raman spectra detector.

The present invention provides a method of detecting multiple gases including obtaining a sample suspected of comprising N₂; and injecting the sample into an N₂ analyzer comprising: a conduit for a gas sample; one or more hollow core photonic crystal fibers (HC-PCF) having a proximal portion and a distal portion, wherein the proximal portion is in communication with the gas sample in the conduit; a laser positioned to strike the gas sample in the conduit and opposite the one or more hollow core photonic crystal fibers; a Raman spectra detector connected to the proximal portion of the hollow core photonic crystal fibers, wherein a Raman spectra is generated when the laser strikes the gas sample that is detected by the Raman spectra detector to detect N₂; a chromatographic detector for O₂, CO₂, and CH₄; and a humidity detector; and a computer processor in communication with the Raman spectra detector, chromatographic detector, and the humidity detector that calculates the relative percentages of each of the multiple gases in the gas sample.

### BRIEF DESCRIPTION OF THE DRAWINGS

For a more complete understanding of the features and advantages of the present invention, reference is now made to the detailed description of the invention along with the accompanying figures and in which:

FIG. 1 is a plot showing interpretation of O₂:CO₂ soil gas concentration data. The trend lines identify the limiting cases of all CO₂ due to methane oxidation or all CO₂ due to biological respiration. Regions delineated by the trend lines indicate the presence of CO₂ dissolution (blue), a combination of the two oxidation processes (green), or excess CO2 from an external source (tan). Data points in the large panel were measured at the Kerr site in Saskatchewan.

FIG. 2 shows data comparing measured values of CO₂ vs N₂/O₂ concentrations plotted along with trend lines for atmospheric gas concentrations, products of biologic respiration and for methane oxidation. The data were taken at the Kerr site in Saskatchewan.

FIG. 3 shows the range of gas concentrations in monitoring wells at the Kerr site. Concentrations were measured by gas chromatography (GC).

FIG. 4 shows an energy level diagram for Raman spectroscopy of N₂.

FIG. 5A is a cross sectional image of the type of hollow core photonic crystal fiber that is used for Raman measurements of soil gases. At least 95% of the light travels through the center bore.

FIG. 5B shows an HC-PCF attenuation spectrum annotated with the wavelengths of an excitation laser (532 nm) and the Raman output wavelengths of CO₂, O₂, N₂, CH₄, and H₂O.

FIG. 6 shows an absorption spectra for 1.5% CO₂ and 1 ppm CH₄ over a 1-m-long optical path.

FIG. 7 is a schematic diagram of Phase I Raman system showing the optical train at right and a detail view of the optical alignment with the hollow core fiber in the inset at left.

FIG. 8 shows transmission curves for bandpass filters that can be used in a Raman detection system. Raman wavelengths of the target gases are indicated.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Soil gas analysis for CCS leak determination is currently performed using gas chromatography (GC). In fact, GC is used for measuring the concentrations of all of the gases listed above except for water vapor. The invention is a replacement for GC measurements of soil gases.

Accurate determination of nitrogen concentrations in soil gas is needed to ensure mass balance in the soil gas analyses that are used for site leak detection. Absent direct measurement of nitrogen concentrations, the accumulated uncertainties in the concentrations of the other gases as determined using commercially available individual gas and humidity sensors can reduce the accuracy of the CCS leak detection method.

The invention can be practiced without requiring consumable supplies such as the bottled helium that is needed for operating a gas chromatograph. This advantage defines a lower cost of operating the gas analyzers and makes implementation of leakage detection more accurate and cost effective. The technology also facilitates real-time continuous automated site monitoring with the potential for data telemetry, which will aid in up-scaling the technology to industrial scales. Continuous monitoring will also give a higher degree of assurance to the public because it will eliminate data gaps, ensuring an arrival of a leakage signal is detected and will demonstrate to stakeholders that monitoring is robust. It will also aid in leakage quantification because it will supply the data density needed for accurate accounting of leakage.

The present invention is a compact, fully automated soil-gas analyzer for monitoring carbon capture and storage sites (CCS). Large scale CCS is a realistic approach to limiting CO₂ emissions from power plants, natural gas wells, and other stationary, high-volume CO₂ sources. As with so many aspects of global warming, CCS generates technical, political, and economic concerns. Issues include cost uncertainties and technical questions about long term efficacy and safety. This fully automated monitoring and verification technology will assuage worries about CCS performance and, therefore, is as an enabling technology for large-scale carbon capture.

Today, most CCS monitoring and verification methods probe the deep geological formations surrounding the capture reservoir. This is not surprising. A great deal of CCS technology is derived from the petroleum industry's use of supercritical CO₂ for oil extraction. Oilfield diagnostic tools—such as seismic imaging and conductivity changes—are then used to monitor deep CCS storage reservoirs. Those living near CCS sites are concerned about possible effects on themselves and their land due to leaks from deep underground reaching the surface. Both issues can be addressed by detecting leaking CO₂ in near-surface soil (the vadose zone).

The present invention makes possible the measurement of gases from samples obtained from locations around CCS sites. Soil gas analysis presents several challenges because biogeochemical processes create short- and long-term variations in local CO₂ concentrations. Carbon dioxide from underground leaks (if any) must be differentiated from the natural, fluctuating background levels of CO₂. Some of the present inventors have recently demonstrated a measurement approach that uses the concentrations of four simple gases—N₂, O₂, CO₂, and CH₄—plus humidity to determine the CO₂ concentrations due to soil biogeochemistry. Using the present invention excess CO₂ can then be attributed to underground leaks. This development is significant because it will lead to quick, decisive leak identification and remediation. The devices and methods of the present invention change the way CCS sites are monitored. Implementing the new leak detection protocol requires stand-alone soil gas analyzers for N₂, O₂, CO₂, and CH₄. Inexpensive methods exist for analyzing O₂, CO₂, CH₄, and water vapor; but, N₂ is a problem. Existing N₂ measurement technology is cumbersome, requires consumable supplies, or is incompatible with O₂ analysis. The present invention includes the use of Raman spectroscopy with hollow core, photonic crystal fibers (HC-PCF) for quantitative concentration measurements of N₂, O₂, CO₂, and CH₄ needed for process-based leak detection. Measurement precision can match that obtained from portable gas chromatographs.

The Raman approach can be tested against gas chromatographic analysis of grab samples obtained from soil-gas monitoring wells CCS sites in, e.g., Cranfield, Miss. and/or Weyburn, Saskatchewan, Canada. The gas analyzer of the present invention can also include an inexpensive, capacitive humidity sensor.

The gas analyzer of the present invention can be a fully-automated, self-contained, and will not require any consumable supplies. Each analyzer would be able to sample gas from multiple depths within a monitoring well. A small solar panel and storage battery can also be included to supply the operating power, and may also include data storage, instrument diagnostics, and wireless capabilities to transmit data via a wireless network.

One example of a location for sampling includes, e.g., CCS sites co-located with large (≧500 MW), coal-burning facilities. There are about 2,300 such power plants world-wide and 20% are on sites that are geologically favorable for CCS. Each site could be fully monitored using an average of, e.g., 2, 3, 4, 5, 6, 7, 8, 9, 10 or more monitoring wells with one analyzer per well.

The present invention has further advantages in that it does not require CO₂ isotope information. Although some researchers expect δ¹³CO₂ to characterize the CO₂ source (soil activity vs. deep storage leaks), some of the present inventors have used such measurements to calibrate and verify our method; therefore, isotope data are not needed but may be used for additional verification if desired. In any case, δ¹³CO₂ measurements are themselves ambiguous for detecting CO₂ leakage due to the overlap in δ¹³CO₂ values between CO₂ produced from soil respiration and CO₂ generated from the combustion of fossil fuels.

Measurement Protocol. Vadose zone measurements of soil gases are important for CCS monitoring because the region is readily accessible and the near-surface gas concentrations are of immediate concern to nearby residents. However, simply measuring near-surface CO₂ concentrations is inadequate for detecting leaks from CCS sites. Observed carbon dioxide concentrations are influenced by microbial and root respiration, CO₂ dissolution into ground water, reactions with carbonates, methane oxidation, and barometric pressure driven mixing with air. All of these effects need to be considered before changes in CO₂ concentrations can be attributed unambiguously to leakage from a geologic carbon storage formation.

The new protocol uses concentrations of N₂, O₂, CO₂, and CH₄ from monitoring wells to promptly distinguish a leakage signal from background CO₂. The approach was developed at a CO₂-rich playa lake and also using a volcanic region that emits carbon dioxide from deep underground as a proxy for leakage from a carbon storage reservoir.

First, measuring the ratio of CO₂ concentration to O₂ provides insight into the amount of CO₂ formed locally by methane oxidation,

CH₄+2O₂→CO₂+H₂O (1)

or biological respiration,

CH₂O+O₂→CO₂+H₂O (2)

where CH₂O is representative of organic material that fuels aerobic microbial respiration.

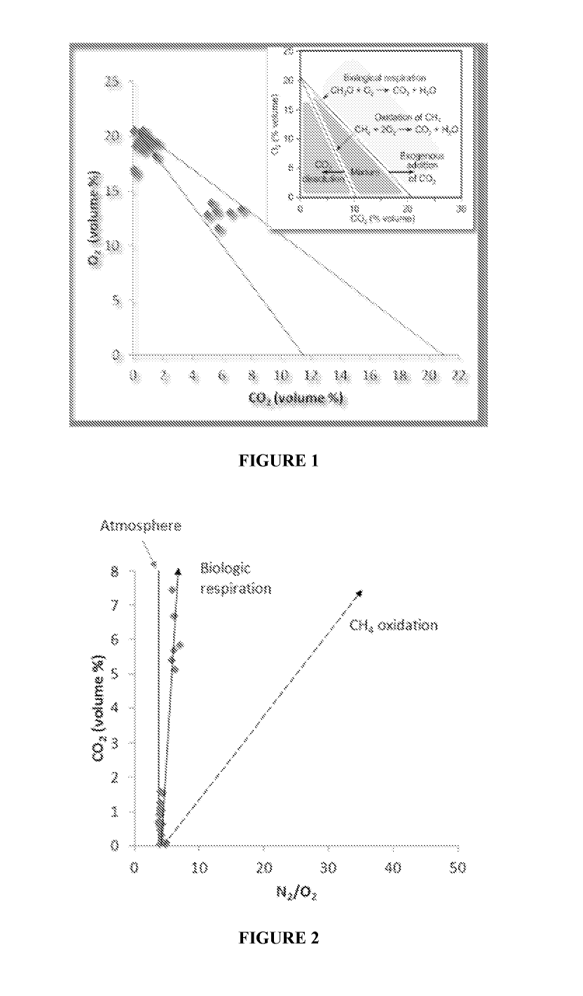

FIG. 1 is a plot showing interpretation of O₂:CO₂ soil gas concentration data. The trend lines identify the limiting cases of all CO₂ due to methane oxidation or all CO₂ due to biological respiration. Regions delineated by the trend lines indicate the presence of CO₂ dissolution (blue), a combination of the two oxidation processes (green), or excess CO₂ from an external source (tan). Data points in the large panel were measured at the Kerr site in Saskatchewan.

The different O₂:CO₂ stoichiometries in Equations (1) and (2) define trend lines that divide data regions into low CO₂ concentrations indicative of carbon dioxide loss due to dissolution (blue region in the inset), a mixture of the two oxidation mechanisms (green), and excess CO₂ due to an external source (yellow).

The data points in the larger panel are measurements obtained by two of the present inventors at the Kerr site in Saskatchewan close to the US-Canadian border, and are part of the evidence used to show that the site was free of carbon dioxide leaking from underground storage.

Next, the ratio of CO₂ to N₂ is altered by either the addition or removal of a gas. Nitrogen enrichment indicates gas removal due to CO₂ dissolution into groundwater. Nitrogen depletion, on the other hand, represents dilution by an added gas and is reason to suspect exogenous CO₂. Nitrogen is a key marker because it is nearly inert. Because of its inert nature, N₂, is difficult to detect, thus the need for a reliable and robust N₂ analysis apparatus and method for use in the field. Extreme denitrification is rare and can be tested, should it be necessary as a follow up to our approach, using GC or mass spectrometry to measure Ar to N₂ ratios.

FIG. 2 shows data comparing measured values of CO₂ vs N₂/O₂ plotted along with trend lines for atmospheric gas concentrations, products of biologic respiration and for methane oxidation. The data were taken at the Kerr site in Saskatchewan.

Next, the ratio of CO₂ to N₂/O₂ determines the amount of CO₂ that might originate from biological processes versus CH₄ oxidation. Large concentrations of carbon dioxide combined with large N₂/O₂ ratios indicate CO₂ formation due to hydrocarbon oxidation, not introduction from other sources. The data and trend lines show how ratio measurements identify oxidation processes. Addition of CO₂ from leaks would be indicated by data points along the vertical line labeled “Atmosphere.” The N₂/O₂ ratio would remain at 3.7 because both gases would be diluted equally.

In the next step of the measurement protocol, CH₄ concentrations are measured to identify relatively large amounts of methane that are either generated from organic matter within wet soil or come from deep sub-surface sources. Concentrations of methane that are well above the atmospheric 1.8 ppm can be oxidized to have a measurable impact on CO₂ concentrations in the sample region.

Finally, correct final measurements for each of the gases have been found to require taking into account humidity, thus the need for humidity measurements. The gas chromatograph used in the previous work is not sensitive to water vapor and data analysis assumed near-saturation conditions for gas in the sample wells. The amount of water vapor, 2.2% for saturated air at 20° C., exceeds target N₂ measurement precision. Therefore, it makes sense to track humidity to prevent variations in water vapor concentrations from adding noise to the Raman measurements.

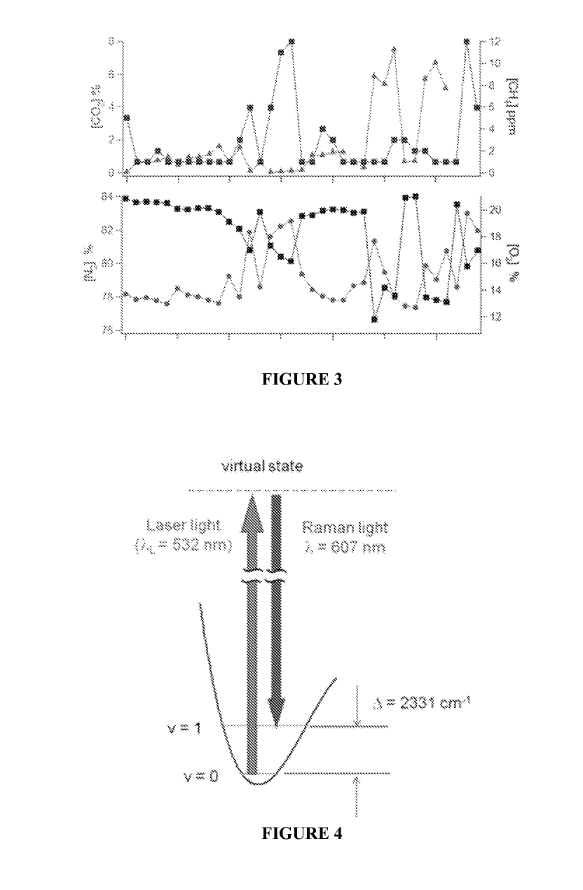

Gas Concentrations. FIG. 3 shows the gas concentrations measured by GC that were reported by two of the present inventors for test wells at multiple locations within the Kerr site in Saskatchewan. These plots show that commercial analyzers should achieve absolute precision of 1% for O₂, 1.5% for N₂, and 1 ppm for CH₄. Although the data imply that 5,000 ppm precision would be suitable for CO₂, more accurate measurements are obtained at 500 ppm, which is close to the ambient atmospheric concentration. By increasing the precision it is possible to achieve finer resolution to distinguish conditions close to standard atmospheric gas concentrations.

The present invention can replace gas chromatography with a gas detection method that includes a Raman spectroscopy detector. The instant invention is made useful by improving on prior Raman spectroscopy methods using hollow-core photonic crystal fibers (HC-PCF) to contain the gas samples. Measurable Raman intensities are about two-orders of magnitude or more larger than is possible using standard Raman measurement techniques. The intensity improvement of the present invention is crucial for successful measurements of nitrogen concentrations and, as a result, for complete mass balance in the gas analysis.

HC-PCF Raman detection with precision equal to or better than the 2% repeatability possible using commercial gas chromatographs (GC). The apparatus depicted in FIG. 6 achieved concentration uncertainties of ±0.15% and ±0.10% for N₂ and O₂, respectively, after 20 seconds of signal averaging. Note that the precision is the relative uncertainty of a concentration measurement and not the absolute concentration error; 2% repeatability of a 79% N₂ concentration is 0.02×0.79=±1.6% absolute concentration.

The HC-PCF Raman gas analysis of the present invention includes a system that records complete Raman spectra using a small optical spectrometer equipped with a low-noise multichannel detector. The spectrometer can be highly effective, but it is expensive. Thus, in one embodiment of the present invention the Raman light is measured using a set of optical bandpass filters and a single channel, photodetector such as an avalanche photodiode.

Raman Spectroscopy. Gas detection methods based on absorption spectroscopy at near- or mid-infrared wavelengths do not work for nitrogen (N₂). While commercial instruments can achieve part-per-billion detection limits for gases such as water vapor, carbon dioxide, carbon monoxide, methane, ammonia, and others, N₂ is absent from the list. However, the present inventors recognized that N₂ can be identified using Raman spectroscopy. In practice, Raman measurements are difficult because the cross sections are so small. The present invention compensates for those small cross sections by using hollow-core fibers to collect Raman light from an extended sample region. Generally, the present invention will use linear Raman spectroscopy, not one of the many types of coherent processes. This is because linear Raman measurements are more straightforward than coherent Raman techniques, and much more robust. Linear Raman signals are directly proportional to the gas concentration and to the laser power.

Raman spectroscopy is a form of vibration spectroscopy in which laser light at wavelength, λ_{L}, drives molecules that are originally in their ground vibrational state (v=0) to a so-called virtual electronic state. The molecule then returns to a different vibrational state by emitting a photon at a longer wavelength than λ_{L}. Conservation of energy dictates that the difference in the optical frequencies between the two wavelengths matches the vibrational energy that remains in the molecule. The wavelength difference between λ_{L} and the emitted Raman light is determined by the molecular vibrational modes. FIG. 4 is an energy level diagram for Raman scattering in N₂ using a green 532 nm laser; 607 nm light is emitted.

Table 1 lists for the gases important to CCS soil gas analysis the Raman shifts, detection wavelengths (for λ_{L}=532 nm), and relative cross sections. For all gases, the Raman output power, P_{R},

$${P}_{R} = {( \frac{{λ}_{0}}{λ} )}^{4} {σ}_{0} {ρP}_{L} {Ω}_{0} 𝓁 \qquad ( 3 )$$

where σ₀ is the molecular Raman cross section in response to laser wavelength λ₀, λ is the laser wavelength used, ρ is the sample density, P_{L} is the laser power at λ, Ω₀ is the solid angle of the light collection optics, and l is the interaction length between the laser beam and the gas sample. The solid angle is important because Raman light is emitted isotropically from the sample; the light is distributed uniformly in all directions and only a small fraction of the total light can be collected. In conventional Raman instruments, the laser beam runs parallel to the entrance slit of a monochromator. Light collection optics image the interaction length, l, onto the entrance slit and the solid angle, Ω₀, is matched to the monochromator numerical aperture (NA). Most monochromators have numerical apertures in the range 0.08≦NA≦0.13. At unit magnification, the interaction length, l, equals the monochromator slit height, h, and matching Ω₀ to the monochromator numerical aperture (NA) gives:

P_{R}=πσρP_{L}h(NA)². (4)

Slit heights, h, are ˜1 cm. When hollow-core photonic crystal fibers are filled with a gas sample, however, the interaction length, l, equals the entire length of the fiber. Equation (4) predicts that a 1-m-long fiber should increase Raman signals by two orders of magnitude over free-space measurements. Chen reported Raman signal enhancements of “several thousand” from a 59-cm-long hollow core PCF.

Table 1 lists for the gases important to CCS soil gas analysis the Raman shifts, detection wavelengths (for λ=532 nm), and relative cross sections. The detection wavelengths in the Table are the wavelengths of the Raman light from gases listed.

TABLE 1
Raman wavelengths and cross sections.
Table 1 - Raman wavelengths and cross sections

| | | Detection | |
|---|---|---|---|
| | Raman shift | Wavelength | Cross Section |
| Compound | (cm⁻¹) | (nm) | (N₂ = 1) |
| Nitrogen | 2330.7 | 607.3 | 1.0 |
| Oxygen | 1554.7 | 580.0 | 1.3 |
| Carbone dioxide | 1285.5 | 571.1 | 0.9 |
| | 1388.3 | 574.4 | 1.4 |
| Methane | 2914 | 629.6 | 6.0 |
| Water vapor | 3651.7 | 660.3 | 2.5 |

Raman Gas Analysis Using Hollow-Core Photonic Crystal Fiber. Hollow-core photonic crystal fibers use a honeycomb-like pattern of air channels to guide light through a central, hollow core. At least 95% of the transmitted light is contained within the central core. Chen and co-workers improved Raman sensitivity over 1000-fold using hollow-core photonic crystal fibers to contain gas samples by exposing the signal over an extended length. Almost all previous work on Raman spectroscopy of nitrogen and oxygen used liquified or high pressure gases to get sufficient density for observation, and some of the earliest experiments on the liquified gases required eight hours of measurement time to get useful spectra.

Much of the signal increase is due to the longer interaction length: l increases from ˜1 cm in the free space case to the nearly the full length of the PCF. Raman light is emitted isotropically (i.e., uniformly in all directions) along every point within the fiber's hollow core. Light emitted within the fiber numerical aperture stays within the fiber core and gets directed to one end or the other. This collection process occurs at every point along the length of the fiber. Raman light exiting from the end of the fibers contains contributions from the entire fiber, yet the spatial properties of the emitted light are determined entirely by the fiber core diameter and the numerical aperture (NA). The light emanates from 6.5-μm-diameter spot (i.e., nearly a point source) into a 24° divergent cone. The Raman light is easily collected, filtered, and directed efficiently onto a detector. This is much more efficient than conventional Raman systems that must collect light from an extended line source.

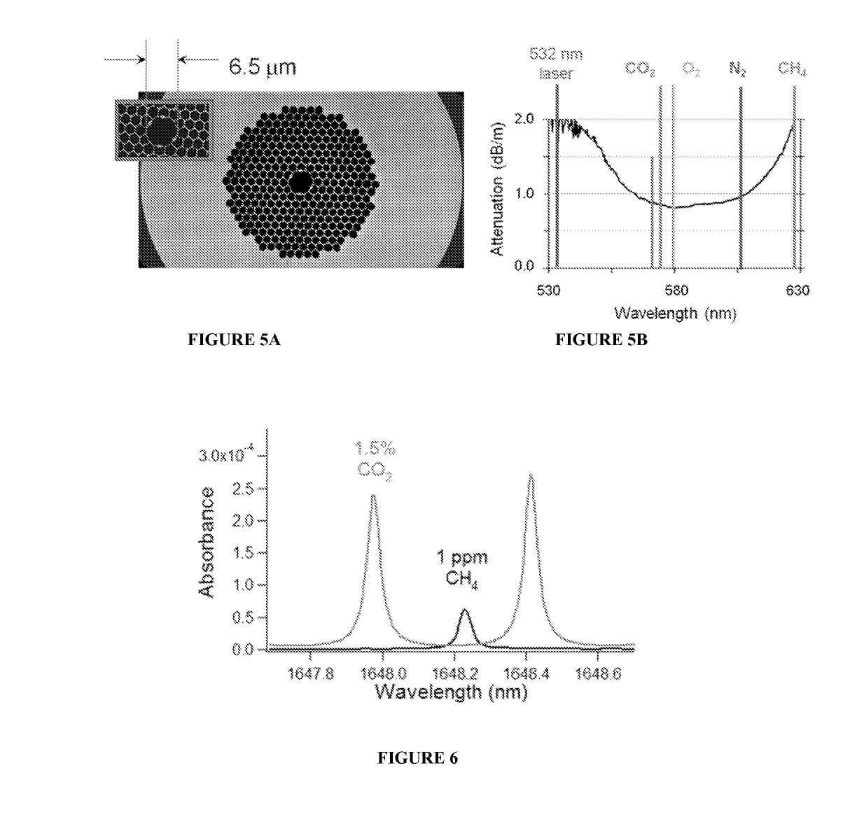

FIG. 5A is a cross-section image of commercial HC-PCF fiber used in demonstration studies. FIG. 5B shows the fiber's attenuation spectrum along with the laser wavelength and the Raman output wavelengths of all gases listed in Table 1. All detection wavelengths except the 660.3 nm light from water vapor lie within regions of low attenuation of the hollow-core photonic crystal fiber. The Raman spectra displayed in the SPIE paper Chen and in U.S. Pat. No. 7,595,882 all omit the wavelength regions that would include Raman signals from carbon dioxide or methane. Neither Chen's patent, nor publication, mentions detecting gases other than homonuclear diatomic gases such as nitrogen and oxygen. FIGS. 5 and 6 in the '882 patent cover Raman shifts between 1950 and 2600 cm⁻¹. This span includes the Raman shifts of oxygen and nitrogen, at 1555 and 2331 cm⁻¹, respectively, but omits the 1285.5 and 1388.3 cm⁻¹ shifts due to carbon dioxide, and the 2914 cm⁻¹ shift due to methane.

Laser light at 532 nm will experience loss at 2 dB m⁻¹ or 37% per m. This is acceptable given the ½ to 1 m fiber lengths anticipated for the proposed gas analyzer. In fact, it was found that the present invention can work even when there is a high loss in the fiber, as shown in FIG. 5B.

Hollow-core PCF fiber is highly flexible and is covered by protective acrylic coating. The fiber can be formed into approximately 2.5 cm diameter without any change in optical properties.

The Raman interaction length in the HC-PCF equals the fiber length, l, and Eq. (4) becomes,

P_{R}=πσρP_{L}l(NA)² (5)

and P_{R} is estimated as 3.3×10⁻¹² W at the N₂ Raman wavelength of 607 nm when P_{L}=50 mW.

The computation is as follows. The N₂ Raman cross section, σ, is 3.8×10⁻³¹ cm² molecule⁻¹ sr⁻¹ for 488 nm excitation. When 532 nm laser light is used, the λ⁻⁴ scaling changes the cross section to 2.9×10⁻³¹. The density of nitrogen is p=2.0×10⁻¹⁹ molecules cm⁻³ at 1 atm and 20° C. Gas can be pushed through or pulled through the fiber by a small pump that imparts a linear pressure (density) change along the length of the fiber. In the case of the pump pulling gas through the fiber the gas pressure typically drops from 1 to 0.75 atm and the average density is be ρ=1.8×10¹⁹ cm⁻³. The laser power is taken to be 50 mW laser (P_{L}) as is available from compact commercial solid state lasers. Applying these values to Eq. (5) and assuming a 1-m-long hollow core fiber gives P_{R}=3.3×10⁻¹² W at the N₂ Raman wavelength of 607 nm; or, 1.0×10⁷ photons per second.

The present invention can use at least two different methods for detecting the Raman light intensity. A first method uses a miniature optical spectrometer (Ocean Optics QE65 Pro). In one embodiment, the Raman light emitted from one end of the hollow core fiber is collimated by a lens, passes through a dichroic mirror that transmits the Raman light and reflects most 532 nm laser light, passes through a notch filter that selectively removes 532 nm light and transmits the Raman light, and is focused into an optical fiber that delivers the light to the spectrometer.

The second detection method uses bandpass filters and an avalanche photodiode (Analog Modules 711-2-3A) that has a gain of 7.7 V/nW and 10 fW Hz^{-1/2} noise. The QE65 Pro spectrometer is replaced with a filter wheel containing bandpass filters at wavelengths chosen to selectively transmit the different Raman wavelengths and to selectively transmit wavelengths that lie between Raman wavelengths. Differential light intensities at the wavelength range containing a Raman wavelength relative to the light intensities at wavelengths between Raman wavelengths can be used to quantify the intensities of Raman light in the presence of a broad spectrum of baseline wavelengths.

Response Times. One more parameter of the present invention is the time-scale for the Raman measurements. Gas flow through a hollow-core PCF is characterized by the Hagen-Poiseuille equation:

$$Q = 1.23 \times {10}^{4} \frac{πΔ {PR}^{4}}{μ 𝓁} \qquad ( 6 )$$

where Q is the flowrate (m³ s⁻¹), ΔP is the pressure drop (atm), R is the hollow core radius (m), μ is the dynamic viscosity of air (kg m⁻¹ s⁻¹), and l is again the interaction length which is the length of the HC-PCF (m). The dynamic viscosity can be taken as independent of pressure for P≦10 bar (10 atm) and is only weakly dependent on temperature over the expected temperature range; μ is 1.8×10⁻⁵ kg m⁻¹ s⁻¹ at 15° C. The gas residence time in the fiber, r (s), is the core volume, V=πR²l, divided by Q: The radius of the hollow core fiber is 3.25 μm (3.25×10⁻⁶ m). Typical parameters are l=1 m and ΔP=0.25 atm, giving τ=568 s or 10 minutes which is acceptable given the slow rate of change of gas concentrations that occur in the vadose zone monitoring wells. The residence time can be reduced by increasing ΔP, reducing the fiber length l, or both.

Measurement of CO₂, CH₄, and Water Vapor. In addition to measuring N₂ and O₂ concentrations, a complete gas analyzer for CCS monitoring requires concentration of carbon dioxide, methane, and water vapor. Well characterized technologies other than the Raman approach can be used to measure one or more of those three gases and the results combined with Raman analysis of the gas sample. Near-infrared absorption spectroscopy with a tunable diode laser can be used to detect CO₂ and CH₄. A near infrared wavelength range where both CH₄ and CO₂ can be measured simultaneously within the wavelength scanning range of a single diode laser in shown in FIG. 6. In other words, both gases can be detected from a single wavelength scan. The wavelength range is also free of interfering absorbances due to water vapor. Concentration detection limits of 0.2 and 600 ppm for methane and carbon dioxide respectively.

Compact inexpensive solid-state and non-dispersive infrared sensors for both CH₄ and CO₂ are available, detection sensitivity for CH₄ is poor and many common hydrocarbons create false positive signals for CH₄. Oxygen can be detected using galvanic sensors. Water vapor can be measured by capacitive sensors. Table 2 lists commercially available sensors for CO₂, O₂, CH₄, and water vapor. However, results of recent soil gas measurements at a CCS site by one of the inventors of the instant invention indicate a site-specific bias in the data comprising significant NDIR sensor error, and a lesser degree of error from galvanic cell technology. Neither NDIR, nor galvanic cell technologies, consistently produced data in the field with the necessary quality to perform process-based monitoring at geologic carbon storage sites. NDIR sensors that had been calibrated successfully by GC in the laboratory showed responses in the field that varied between 1.37 and 1.85 times the calibrated values. The galvanic O₂ sensor consistently under-reported the O₂ concentration. No obvious reason for the bias was identified, although similar results have been reported in other research.

TABLE 2
Commercial Gas Sensors

| | | | | | | |
|---|---|---|---|---|---|---|
| | | | Response | Environmental | | |
| Sensor Type | Range | Accuracy | Time | Limits | Technology | Manufacturer |

CO₂

| | | | | | | |
|---|---|---|---|---|---|---|
| GMT 222 | 0 to | ±1.5% of | 30 secs | −5° C. to +45° C. | NDIR | Vaisala |
| | 2000 ppm | range + 2% | | 0 to 85% RH | | |
| | | of reading | | | | |
| GMT 221 | 0% to | ±1.5% of | 20 secs | 0° C. to 50° C. | NDIR | Vaisala |
| | 20% | range + 2% | | 90% RH | | |
| | | of reading | | | | |

O₂

| | | | | | | |
|---|---|---|---|---|---|---|
| Series 2000 | 0% to | ±1% | <30 secs | 0° C. to 50° C. | Electro- | Alpha |
| | 25% | | | no RH spec | chemical | Omega |

CH₄

| | | | | | | |
|---|---|---|---|---|---|---|
| MSH-P-HR | to 10% | 100 ppm | 30 secs | −25 to 50° C. | NDIR | Dynament |
| | 10 to | 1000 ppm | | 0 to 95% RH | | |
| | 100% | | | | | |

Humidity

| | | | | | | |
|---|---|---|---|---|---|---|
| HUMICAP180R | 0 to | 1.5% RH | 8 secs | −40 to +80° C. | Capacitive | Vaisala |
| Humidity | 100% RH | | | | thin-film | |
| | | | | | polymer | |

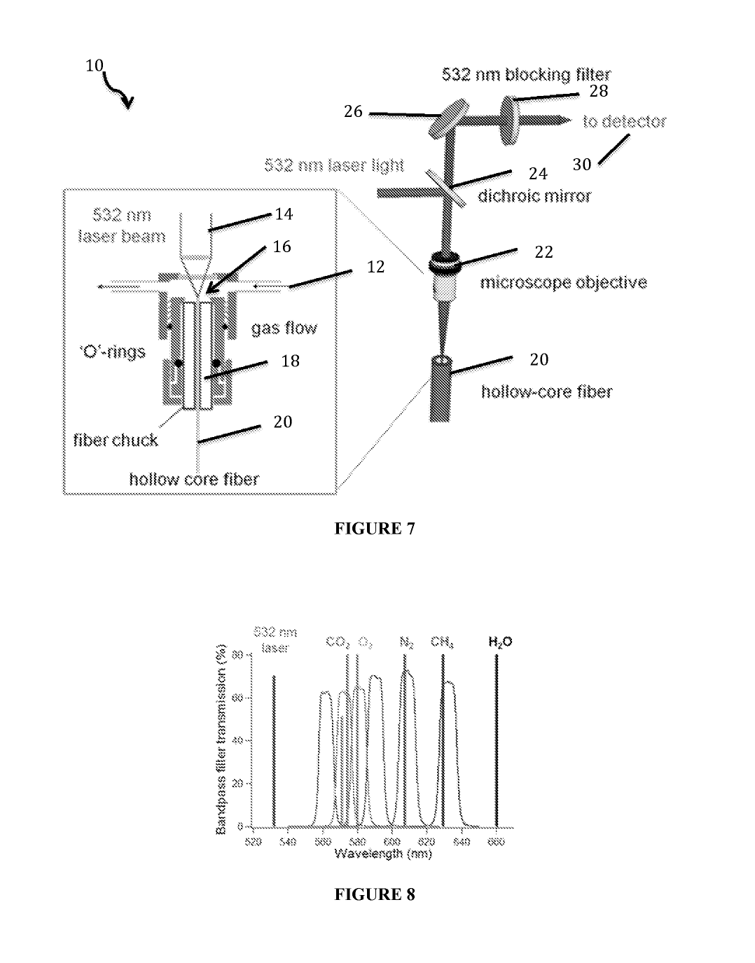

FIG. 7 is a schematic diagram of the apparatus 10. A HC-PCF-Raman system is shows using a 532 nm solid-state laser 14 (e.g., a Thorlabs) capable of 50 mW continuous output. The laser 14 has excellent spatial mode quality, high stability, and a narrow linewidth (<0.03 nm). A conduit 12 is shown that includes a gas 16, which is sealed using a fiber chuck 18 that brings a proximal portion of the hollow core fiber 20 in contact with the gas 16. These optical properties are important for efficient coupling of laser 14 light into the fiber 20. The laser beam reflects from a dichroic mirror 24 and then is focused into one end of the hollow-core fiber using a microscope object 22 having a numerical aperture (0.2) matched to the fiber 20. Backward traveling Raman light passes upward through the dichroic mirror 26 and then through a blocking filter 28 that removes residual scattered 532 nm light before entering a detector 30.

For ease of detector alignment, a conventional optical fiber is used to transmit the collected light to the detector 30.

The detector can be a small commercial spectrometer (e.g., an Ocean Optics QE65 Pro). The spectrometer sensitivity and detector noise properties are described below. The QE65 Pro is configured with a 1,200 line per mm grating that has optimum performance between 400 and 800 nm. When equipped with a 25 μm entrance slit, the wavelength resolution, 0.5 nm, corresponds to 13 cm⁻¹ at the N₂ Raman line, matching the spectrometer resolution used by Chen. In one non-limiting example, the spectra were measured using a 200 micron diameter collection fiber with a 100 micron spectrometer slit.

Most of the gas sample flows through the headspace region. A small amount is pulled through the hollow core fiber using a diaphragm pump (not shown) at the fiber exit. Gas is measured by an MKS Series 910 DualTrans pressure gauge. Alternatively, the gas could be pushed into the headspace region using a pump.

Raman spectra of pure N₂ and pure O₂ can be acquired to determine the instrument response, wavelength resolution, and signal-to-noise ratios. Signal magnitudes can be compared the photon budget calculations. Calibration curves are generated using prepared, flowing mixtures of the two gases. These curves are used to test measurement linearity. Raman signal intensities can be compared with variations in laser intensity to determine any unexpected non-linearities that might affect performance. Step changes in concentrations can be used to determine the actual gas residence time in the fiber and compare that time with the response time calculations.

Allan variance analysis can be used to determine the improvements in signal-to-noise ratios as a function of signal averaging times and to identify the characteristic time scales for a non-statistical (systematic) noise sources. These measurements define the measurement capabilities of the apparatus.

In one example, the apparatus uses a 40 mW, 532-nm laser, a spectrometer (Ocean Optics), gas handling hardware, including: electronic flow controllers, vacuum gauge, optical mounts, an optical breadboard, and software to operate the spectrometers and to collect and analyze the resulting spectra. Additional equipment includes hollow-core photonic crystal fibers (HC-PCF), optics, optical mounting hardware, and as may be required for calibration and other purposes one or more gas mixtures.

Detection of the Raman light using a combination of bandpass filters in place of the spectrometer. In another example, the spectrometer can be replaced with a set of optical bandpass filters and a single-element, Si avalanche photodiode. FIG. 8 shows the transmission curves of the six commercial bandpass filters that can be used. In one specific example, three optical bandpass filters can be used: the two filters centered on the O₂ and N₂ Raman lines, plus one filter centered at 590 nm.

The detector can be an avalanche photodiode plus preamplifier module from Analog Modules (Longwood, Fla.) that has a 0.5 mm diameter detector element, 7.7 V/nW response, 9 kHz electronic bandwidth, and 10 fW Hz^{-1/2} noise. Initial characterization of the detector will use an electronic spectrum analyzer to look for any frequency-dependent noise and to identify optimum frequency bands, if any. Raman and background signals will be recorded using a mechanical chopper and lock-in detector as the different bandpass filters are placed into the optical path.

The spectrometer is more effective than the bandpass filter approach for identifying light from unexpected sources and for determining background light due to fluorescence or a similar broadband emission. The skilled artisan will recognize that there are tradeoffs between analyzer cost and performance, and between cost and mechanical complexity. The spectrometer has the advantages of versatility and complete solid-state construction, however, spectrometers are generally more expensive than the bandpass filters and single-element detector. On the other hand, using a standard filter wheel adds a mechanical feature that may need to be carefully engineered replaced with an electronic equivalent.

The response to binary mixtures of nitrogen and oxygen show the Raman technique to be linear with gas concentration. No cross-talk is observed, meaning that Raman light from one analyzed gas does not give a false positive signal for a different gas.

Allan variance analysis can be used to identify the limiting noise source(s) and to determine the best possible measurement precision that can be obtained from signal averaging.

The HC-PCF Raman apparatus can be used for analysis of gas concentrations in grab samples acquired from CCS site monitoring wells.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present invention may also include methods and compositions in which the transition phrase “consisting essentially of” or “consisting of” may also be used.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A gas analyzer comprising:
a conduit for a gas sample;
one or more hollow core photonic crystal fibers (HC-PCF) having a proximal portion and a distal portion, wherein the proximal portion is in communication with the gas sample in the conduit;
a laser positioned to strike the gas sample in the conduit and opposite the one or more hollow core photonic crystal fibers; and
a Raman spectra detector connected to the proximal portion of the hollow core photonic crystal fibers, wherein a Raman spectrum is generated when the laser strikes the gas sample that is detected by the Raman spectra detector without regard to the level of attenuation.

2. The analyzer of claim 1, wherein the detector is a spectrometer.

3. The analyzer of claim 1, wherein the Raman spectrometer is adapted for linear Raman spectroscopy.

4. The analyzer of claim 1, wherein the detector comprises one or more avalanche photodiodes.

5. The analyzer of claim 1, wherein the gas is at a pressure at or about atmospheric pressure, above atmospheric pressure, or below atmospheric pressure.

6. The analyzer of claim 1, further comprising at least one of a near-infrared analyzer for CO₂ and CH₄, and a capacitive humidity sensor in fluid communication with the gas sample.

7. The analyzer of claim 1, wherein the analyzer does not require any consumable supplies.

8. The analyzer of claim 1, further comprising a microscope objective between the laser and the Raman detector.

9. The analyzer of claim 1, further comprising at least one of a power source, a small solar panel, a storage battery, data storage, instrument diagnostics, and wireless capabilities to transmit data via a wireless network.

10. A method of for detecting a gas comprising:
obtaining a sample suspected of comprising N₂; and
injecting the sample into a gas analyzed comprising a conduit for a gas sample, one or more hollow core photonic crystal fibers (HC-PCF) having a proximal portion and a distal portion, wherein the proximal portion is in communication with the gas sample in the conduit, a laser positioned to strike the gas sample in the conduit and opposite the one or more hollow core photonic crystal fibers, and a Raman spectra detector connected to the proximal portion of the hollow core photonic crystal fibers, wherein a Raman spectra is generated when the laser strikes the gas sample that is detected by the Raman spectra detector; and
measuring at least one of N₂, O₂, CO₂, and CH₄ and H₂O.

11. The method of claim 10, wherein the Raman spectra detector is a Raman spectrometer.

12. The method of claim 10, wherein the Raman spectra detector is a linear Raman spectroscopy.

13. The method of claim 10, wherein the Raman spectra detector comprises one or more avalanche photodiodes.

14. The method of claim 10, wherein the gas is not at a pressure above atmospheric pressure.

15. The method of claim 10, further comprising the step of positioning at least one of a near-infrared analyzer for CO₂ and CH₄, and a capacitive humidity sensor in fluid communication with the gas sample.

16. The method of claim 10, wherein the analyzer does not require any consumable supplies.

17. The method of claim 10, further comprising the step of positioning a microscope objective between the laser and the Raman spectra detector.

18. The method of claim 10, further comprising at least one of a power source, a small solar panel, a storage battery, data storage, instrument diagnostics, and wireless capabilities to transmit N₂, O₂, CO₂, and CH₄ and H₂O data via a wireless network.

19. The method of claim 10, further comprising the step of measuring at least one of N₂, O₂, CO₂, and CH₄ and H₂O from one or more monitoring locations to distinguish a leakage signal from background CO₂.

20. The method of claim 10, wherein N₂ enrichment in the sample is indicative of gas removal due to CO₂ dissolution into groundwater, whereas N₂ depletion is indicative of dilution by an added gas from exogenous CO₂.

21. The method of claim 10, further comprising the step of determining the ratio of CO₂ to N₂/O₂ to determine the amount of CO₂ that originated from biological processes versus CH₄ oxidation.

22. A method of detecting multiple gases comprising:
obtaining a sample suspected of comprising N₂; and
injecting the sample into a multi-gas analyzer comprising a conduit for a gas sample, one or more hollow core photonic crystal fibers (HC-PCF) having a proximal portion and a distal portion, wherein the proximal portion is in communication with the gas sample in the conduit, a laser positioned to strike the gas sample in the conduit and opposite the one or more hollow core photonic crystal fibers, a Raman spectra detector connected to the proximal portion of the hollow core photonic crystal fibers, wherein a Raman spectra is generated when the laser strikes the gas sample that is detected by the Raman spectra detector to detect N₂, a chromatographic detector for, O₂, CO₂, and CH₄, and a humidity detector, and a computer processor in communication with the Raman spectra detector, chromatographic detector, and the humidity detector that calculates the relative percentages of each of the multiple gases in the gas sample;
measuring at least one gas value of N₂, H₂, O₂, CO₂, CH₄ and H₂O; and
calculating the relative percentages of each of the multiple gases in the gas sample form the at least one gas value.
